(19) United States
(12) Patent Application Publication
(10) Pub. No.: US 2009/0238782 A1
(43) Pub. Date: Sep. 24, 2009

(54) COSMETIC COMPOSITION WHICH INCLUDES AT LEAST ONE POLYSACCHARIDE DERIVED FROM BACTERIA OF HYDROTHERMAL ORIGIN

(75) Inventors: Anne Marie Vacher, Le Chesnay (FR); Marie-Claire Fritsch, Paris (FR)

Correspondence Address: BROWDY AND NEIMARK, P.L.L.C.;624 NINTH STREET, NW, SUITE 300, WASHINGTON, DC, 20001-5303, US

(73) Assignee: LANATECH LABORATOIRE NATURE ET TECHNIQUE, Paris (FR)

(21) Appl. No.: 12/472,181

(22) Filed: May 26, 2009

Related U.S. Application Data
(62) Division of application No. 09/354,570, filed on Jul. 16, 1999, now abandoned.

(30) Foreign Application Priority Data
Jul. 31, 1998 (FR) 9810034

(51) Int. Cl. A61K 8/73; A61K 31/715; A61P 17/00; A61P 17/10; A61Q 17/04

(52) U.S. Cl. 424/59; 424/780

(57) ABSTRACT

The invention relates to a cosmetic composition which includes at least one polysaccharide derived from bacteria of hydrothermal origin. This polysaccharide can consist of an exopolysaccharide which is derived from fermenting the said bacteria. This composition can be applied, in particular, to everyday care products for all types of skin.

The present invention relates to a cosmetic composition which includes at least one polysaccharide which is derived from bacteria of hydrothermal origin.

The invention results from the study, which was carried out by the applicant, of a very distinctive collection of bacteria, namely mesophilic bacteria of hydrothermal origin.

In a general manner, it is known that, in the 1970s, oceanographic biologists participating in deep-sea oceanographic expeditions discovered, to their great surprise, the existence of novel ecological communities which were living in the vicinity of hydrothermal vents. These hydrothermal springs, which are peculiar to active oceanic ridges, originate from the infiltration of seawater into the network of faults which cause it to circulate within the layer of the earth's crust, in proximity to the magma. Whereas the various expeditions in the past had demonstrated that the fauna was not very abundant beyond a depth of 2500 meters, the scientists were surprised to discover an exuberant fauna of mollusks, worms and crustaceans, as well as complex associations of bacteria and invertebrates, around these hydrothermal springs.

A substantial collection of bacteria has been put together gradually, as these expeditions have taken place. These species have been described and, although the majority, which are not pathogenic, belong to known genera, the species are nevertheless novel. Some of these bacteria, which live and reproduce at very great depths and under extreme conditions, have been found to be able to grow under laboratory culture conditions and to synthesize, and to secrete into the culture medium, a number of molecules which are of very great interest to study.

Furthermore, it is well known that some polysaccharides which are extracted from fungi, algae, yeast walls or terrestrial bacteria are known in medical therapeutics for their stimulatory action on macrophages, whose bactericidal and tumoricidal action they improve. These molecules are therefore able to stimulate the immune system, thereby making it possible to prevent a number of ailments.

Some of these polysaccharides, which are extracted from the cell walls of various organisms or microorganisms, therefore display interesting biological activities. It has been possible in part to exploit these biological properties in the cosmetic field. It was shown, for example, that a β-glucan which was extracted from the wall of a yeast, i.e. Saccharomyces cerevisiae, enabled skin to regenerate.

More precisely, the studies conducted by the applicant were aimed at determining the activity, in the cosmetic field, of exopolysaccharides derived from fermenting mesophilic bacteria of hydrothermal origin.

These polysaccharides, which are synthesized by these bacteria which are cultured in the laboratory, are polymers of high molecular weight (from 100,000 daltons to more than a million daltons). They consist of chains of various neutral or acid sugars, with the basic monomeric unit generally representing 4 to 10 residues. While some are linear, most are branched. Others have a structure which is entirely unknown at present.

For the purpose of the study, the applicant studied the efficacy of three polysaccharides which are very different from each other from a chemical point of view and which are derived from fermenting three distinct species of mesophilic bacteria of hydrothermal origin, namely.
two exopolysaccharides, i.e. POL.1 and POL.2, consist of native (that is unmodified) polymers having different neutral sugar/acid sugar ratios; these two exopolysaccharides have very high molecular weights (500,000 to 1 million),
a modified polysaccharide, i.e. POL.3, which was chemically sulfated and then depolymerized, and which has a much lower molecular weight.

With regard to these studies, it is first of all appropriate to recall that the skin constitutes the very first barrier of the body to external attack. The keratinocytes, which make up the majority of the cells of the epidermis, are involved in an extremely precise program of differentiation and maturation which is subjected to numerous interactions between the epidermal and dermal compartments. This process results in the elaboration of keratins and complex lipids which guarantee the role and integrity of the epidermis and of its “barrier” component: the corneal layer. This is the classical and well-known role of the keratinocytes in providing mechanical protection.

What is much more interesting is the involvement of the same cells in the process of cutaneous immune defense. It is nowadays a demonstrated fact that the keratinocytes are the main source, in the epidermis, of mediators of cell communication. They are nowadays recognized, in the same way as the Langerhans cells, as essential and fundamental participants in this defense system. The different cells of the skin act in perfect harmony due to a very elaborate system of intercellular communication which is made up, inter alia, of the cytokine network. The cytokines play a major role in maintaining a normal immune state and are also involved in inflammation, wound healing, angiogenesis, allergy and apoptosis. The regulated expression of each of the cytokines makes it possible to maintain local cell proliferation and metabolism in a state of equilibrium.

Chemical or physical attacks (ultraviolet irradiation, infectious agents, etc.) are able to disturb this equilibrium. This results in the long term in a damaged, desiccated and irritated skin which is prematurely aged and which, furthermore, possesses less and less ability to ensure its own defense and its role as a protective barrier.

This is the reason for the interest, in cosmetics, in seeking to improve the system for defending and protecting the cells of the skin. This modulation of the cutaneous immune defense system can be obtained by the topical application of molecules which are able to stimulate the keratinocytes, thereby specifically inducing the expression of particular cytokines. This results in an arousal of the immune defense system of the skin, which is then able to react more vigorously and rapidly to external attack.

The research studies carried out by the applicant were aimed at demonstrating a possible stimulatory effect of these polysaccharides on the expression, by the keratinocytes, of interleukin 1α. Interleukin 1α is the very first cell communication mediator, which is synthesized and secreted by the keratinocytes and is able to initiate the “immune cascade”.

These activation properties were evaluated on human keratinocytes in primary cultures is using the three different experimental protocols which follow:

### Protocol 1 (Preventive Effect on Keratinocytes):

Carried out on cultured human keratinocytes. Preincubation for 3 hours with the products to be tested, then stimulation with phorbol myristate acetate (PMA). Assay of IL-1α by an immunoenzymic test (values in pg/ml) at the times T1h, T6h and T24h following stimulation. The results are given in Table I below.

TABLE I

| Concentrations | | | | | |
|---|---|---|---|---|---|
| | | | | 5 | |
| 0 | 0.1. μg/ml | 0.5 μg/ml | 1 μg/ml | μg/ml | 10 μg/ml |

| | | | | | | |
|---|---|---|---|---|---|---|
| POL. 1-T1 h | 48 | 50 | 46 | 26 | 17 | 36 |
| POL. 1-T6 h | 34 | 68 | 48 | 86 | 191 | 72 |
| POL. 1-T24 h | 55 | 31 | 65 | 38 | 79 | 36 |
| POL. 2-T1 h | 48 | 47 | 33 | 40 | 26 | 21 |
| POL. 2-T6 h | 34 | 44 | 46 | 149 | 88 | 54 |
| POL. 2-T24 h | 55 | 25 | 24 | 25 | 60 | 49 |
| POL. 3-T1 h | 3 | 16 | 8 | 115 | 37 | 33 |
| POL. 3-T6 h | 40 | 43 | 69 | 77 | 78 | 75 |
| POL. 3-T24 h | 54 | 60 | 24 | 64 | 93 | 75 |

Protocol 2: Carried out on cultured human keratinocytes. Preincubation for 24 hours with the products to be tested, then costimulation with products to be tested/PMA. Assay of IL-1α (values in pg/ml) at time T48h following stimulation. The results are given in Table II below:

TABLE II

| | Concentrations | | | | | |
|---|---|---|---|---|---|---|
| | | | | 5 | | |
| | 0 | 0.5 μg/ml | 1 μg/ml | μg/ml | 10 μg/ml | 50 μg/ml |
| POL. 1-T48 h | 41 | 86 | 64 | 28 | 29 | 28 |
| POL. 2-T48 h | 41 | 33 | 35 | 24 | 21 | 31 |
| POL. 3-T48 h | 41 | 31 | 27 | 29 | 34 | 25 |

### Protocol 3 (Curative Effect on Keratinocytes):

Carried out on cultured human keratinocytes. Costimulation with products to be tested/PMA. Assay of IL-1α (values in pg/ml) at times T1h, T3h and T6h following stimulation. The results are given in Table III below:

TABLE III

| | Concentrations | | | | |
|---|---|---|---|---|---|
| | 0 | 0.1 μg/ml | 0.5 μg/ml | 1 μg/ml | 5 μg/ml |
| POL. 1-T1 h | 62 | 86 | 93 | 52 | 47 |
| POL. 1-T3 h | 67 | 43 | 65 | 47 | 66 |
| POL. 1-T6 h | 30 | 29 | 31 | 45 | 34 |
| POL. 2-T1 h | 62 | 64 | 106 | 56 | 46 |
| POL. 2-T3 h | 67 | 53 | 54 | 48 | 46 |
| POL. 2-T6 h | 30 | 33 | 40 | 60 | 39 |
| POL. 3-T1 h | 62 | 46 | 51 | 52 | 43 |
| POL. 3-T3 h | 67 | 54 | 56 | 53 | 44 |
| POL. 3-T6 h | 30 | 38 | 48 | 61 | 46 |

The data acquired when these different study protocols were carried out enable the conclusion to be drawn that the 3 polysaccharides tested have a stimulatory action on the expression of interleukin la by the cultured human keratinocytes. Furthermore, these products do not exhibit any cell toxicity at the doses tested.

In this in vitro experimental model, these three polysaccharides, which are of greatly differing chemical natures, therefore exhibit an incontestable biological activity. Fortified by this finding, the applicant then sought to demonstrate the positive consequences for the skin of such a biological activity. A variety of studies were therefore carried out in vitro, on cultured keratinocytes, ex vivo, on human skin explants, or in vivo, on animal models. This was done with the aim of demonstrating a possible protective effect of the tested polysaccharides in the face of various attacks on the skin.

The cytotoxicity of the polysaccharides POL.1, POL.2 and POL.3 was studied in regard to a microorganism, typically Candida albicans, using cultured human keratinocytes.

It may be recalled, in this regard, that, under certain circumstances, keratinocytes possess a phagocytic capacity which enables them to participate very actively in the body's struggle against microbial attack. It has been demonstrated in vitro that human keratinocytes are able to destroy a microorganism: Candida albicans. The mechanism of action very certainly involves molecules which are secreted by the keratinocyte against the foreign entity. There is therefore no true phagocytosis of the microorganism; instead, the keratinocyte exhibits cytotoxicity toward it. The keratinocyte is then able to ingest and eliminate the debris of the killed cells.

The skin is subjected daily to attack by microbes of every variety: yeasts, molds, microscopic fungi, bacteria, etc. Some organisms are harmless to the skin, others, which are beneficial to it, participate in equilibrating the saprophytic flora of the skin, while others, finally, are pathogenic. The growth of pathogenic organisms on the skin surface obviously leads to a disequilibrium of the skin flora and to the appearance of more of less serious skin pathologies which require medical treatment.

This explains the interest, in cosmetics, in seeking to prevent this type of problem by attempting to help the skin to defend itself more vigorously and more rapidly against any incipient microbial attack. The applicant therefore sought to demonstrate the stimulatory action of the polysaccharides on the ability of the keratinocytes to destroy various microorganisms which are harmful to the skin.

The toxicity against Candida was measured using a modification of the method of Lehrer & Cline (Interaction of Candida albicans with human leukocytes & serum, J. Bacteriol. 1969:98:996). A suspension of Candida albicans (4×10⁷ 0656CBS Delf cells in PBS) was incubated in the presence of the keratinocyte suspension and in the presence or absence of various doses of polysaccharides to be tested. The incubation was carried out at 37° C. for one hour. After centrifugation, the Candida albicans cells were extracted following addition of Triton X 100 and were stained with a 0.1% solution of methylene blue. The percentage of killed Candida albicans cells (which are stained uniformly) was determined under a microscope and the results were expressed in percentage lyzed.

Products to be tested: pure polysaccharides which were dissolved in the test buffer (PBS); concentrations in μg/ml.

The results, expressed in percentage of microorganisms killed, are given in Table IV below:

TABLE IV

Concentrations

| | 0 | 0.08 | 0.17 | 0.33 | 0.83 | 1.67 |
|---|---|---|---|---|---|---|
| POL. 1 | 2.5 | 4.3 | 7.2 | 10.3 | 12.5 | 12.8 |
| POL. 2 | 5 | 6.1 | 9.9 | 13.0 | 16.3 | 16.4 |
| POL. 3 | 4.9 | 6.8 | 10.8 | 14.4 | 14.8 | 17.0 |

In conclusion, the three products tested exhibit a worthwhile stimulatory activity on the ability of cultured human keratinocytes to destroy this microorganism Candida albicans. However, the polysaccharide POL.1 is less active in this model than are the other two molecules.

The applicant also carried out studies of cytotoxicity with regard to the main organisms involved in acne (Propionibacterium acnes, Propionibacterium granulosum and Staphylococcus epidermidis).

Thus, it is known that young skins, i.e. skins with a tendency to be greasy, are the breeding ground of preference for these microorganisms, which proliferate anarchically in the presence of the excess of sebum which characterizes this type of skin. This represents a vicious circle (excess of sebum, growth of microorganisms, acne, increased production of sebum, etc.) which cosmetic care must attempt to break. Since the tested polysaccharides demonstrated a stimulatory activity on the ability of the epidermal cells to destroy Candida, the applicant studied the effect of these polysaccharides on the 3 main organisms involved in acne. However, there was nothing to suggest that it would be possible to demonstrate an activity on these organisms.

A first study failed to demonstrate any direct bactericidal action of the polysaccharides on the 3 organisms in question. An indirect effect (action of the molecules secreted by the stimulated keratinocyte) was therefore sought.

To this end, the applicant carried out an evaluation of the cytotoxicity-inducing effects of the pure polysaccharides with regard to Propionibacterium acnes, Propionibacterium granulosum and Staphylococcus epidermidis using the supernatants from cultures of human keratinocytes which were incubated with different doses of the products to be tested.

More precisely, three doses, of 0.5 μg/ml, 1 μg/ml and 5 μg/ml, respectively, were tested in accordance with a process which comprised:
incubating the polysaccharides with keratinocyte cultures (3 incubation times of 15 min, 1 h and 6 h, respectively),
recovering the supernatant and diluting it with culture medium,
carrying out successive dilutions,
seeding the microorganism strains on agar, and
determining the MIC (minimum inhibitory concentration).

The products to be tested/keratinocyte incubation time which appears to be most appropriate is the time of 1 hour.

The least diluted culture supernatant solutions are observed to have a bactericidal effect. While the three polysaccharides exhibit an indirect activity on Propionibacterium acnes and Staphylococcus epidermidis, they on the other hand do not exhibit any activity, at the concentrations tested, on Propionibacterium granulosum.

In conclusion, this study demonstrates that the polysaccharides to be tested have an indirect destructive action on two of the main organisms involved in the phenomenon of acne. In this case, too, these polysaccharides are found to be an invaluable aid for the skin, enabling it to regulate and equilibrate its surface microbial flora.

The applicant furthermore carried out an evaluation of the protective effects of 1% aqueous solutions of the polysaccharides (tested at concentrations of from 0.5% to 5%) with regard to the Langerhans cells in cultured human skin explants exposed to UVB irradiation.

It may be recalled that the Langerhans cells, which are dendritic cells situated in the epidermis, are important participants in the immune defense system of the skin. Since they lack melanin, these cells have little protection against attack by ultraviolet light. They therefore constitute a very sensitive parameter for detecting deleterious effects of these radiations.

The skin explants, which were of 8 mm in diameter and which were removed from an abdominal plasty carried out on a 26-year old woman, were cultured in 24-well plates, containing 300 μl of medium per well, with the epidermal surface upwards.

The positive reference consisted of a protective mixture composed of vitamin C and reduced glutathione in aqueous solution.

Application to the surfaces of the epidermes to be treated was repeated at times T24h and T48h.

At time T72h, the explants were subjected to UVB irradiation (total irradiation of 1.5 J/cm²)

At time T96 h, the explants were frozen and transverse sections were cut. These sections were treated with an anti-CD1a (specific marker of Langerhans cells) antibody and then examined in an epifluorescence microscope, when the fluorescent Langerhans cells were counted (counting of 6 fields per slide, that is 12 values per treatment).

The results of this assessment are reported in Tables V and VI below (with Table VI indicating the percentage protection):

TABLE V

| | | | | | | | | | | | % |
|---|---|---|---|---|---|---|---|---|---|---|---|
| | | Ex- | | | | | | | | | via- |
| | Product | plant | Number of cells/field | | | | | | Mean | σ | bility |
| −UVB | Control | A | 51 | 58 | 54 | 49 | 53 | 55 | 57 | 4 | 100 |
| | | B | 64 | 60 | 57 | 59 | 58 | 62 | | | |
| +UVB | Control | A | 30 | 23 | 17 | 27 | 21 | 24 | 26 | 5 | 46 |
| | | B | 28 | 32 | 21 | 36 | 27 | 24 | | | |
| +UVB | Ref. ⊕ | A | 44 | 48 | 44 | 41 | 43 | 47 | 42 | 4 | 73 |
| | | B | 39 | 41 | 34 | 40 | 37 | 40 | | | |
| +UVB | 0.5% | A | 29 | 32 | 37 | 34 | 32 | 31 | 36 | 5 | 63 |
| | POL. 1 | B | 46 | 45 | 32 | 36 | 40 | 35 | | | |
| +UVB | 5% | A | 49 | 53 | 56 | 56 | 50 | 57 | 50 | 5 | 88 |
| | POL. 1 | B | 52 | 47 | 42 | 49 | 43 | 46 | | | |
| +UVB | 0.5% | A | 44 | 52 | 43 | 39 | 47 | 51 | 45 | 4 | 79 |
| | POL. 2 | B | 43 | 46 | 39 | 41 | 46 | 44 | | | |
| +UVB | 5% | A | 25 | 24 | 28 | 30 | 25 | 29 | 27 | 2 | 48 |
| | POL. 2 | B | 30 | 26 | 27 | 26 | 24 | 31 | | | |
| +UVB | 0.5% | A | 32 | 37 | 37 | 41 | 39 | 35 | 36 | 3 | 63 |
| | POL. 3 | B | 30 | 38 | 36 | 32 | 33 | 37 | | | |
| +UVB | C5% | A | 42 | 38 | 47 | 43 | 45 | 40 | 45 | 4 | 80 |
| | POL. 3 | B | 52 | 49 | 44 | 49 | 51 | 42 | | | |

TABLE VI

| | | | Mean number | % |
|---|---|---|---|---|
| | Product | Concentration | of cells | protection |
| −UVB | Control | 0 | 57 | 100 |
| +UVB | Control | 0 | 26 | 0 |
| +UVB | Ref. ⊕ | 100 μg/ml | 42 | 51 |
| +UVB | POL. 1 | 0.5% | 36 | 32 |
| | solution | 5% | 50 | 78 |
| +UVB | POL. 2 | 0.5% | 45 | 61 |
| | solution | 5% | 27 | 4 |
| +UVB | POL. 3 | 0.5% | 36 | 32 |
| | solution | 5% | 45 | 63 |

In conclusion, this study, which was carried out on human skin explants, demonstrates that the polysaccharides have a good protective effect. This protection is particularly strong in the case of the No. 1 and No. 3 polysaccharides. On the other hand, polysaccharide No. 2, which exhibits an excellent protective effect at the dose of 0.5% (of 1% solution), is found to have a cytotoxic effect at the 5% dose. This phenomenon is without doubt due to a joint UVB/high concentration of POL.2 cytotoxic action.

This demonstration, which was carried out in an experimental model which was very close to the in-vivo situation, does not allow any conclusion to be drawn with regard to the mechanism involved in the observed protection phenomenon. However, the large number of data acquired, taken overall, enables the applicant to suppose a mechanism of protecting the Langerhans cells which is both direct and indirect:
direct, anti-free radical protection (since the Langerhans cells are extremely sensitive to attack by the free radicals which are induced by UV radiation).
indirect protection—improvement of the mechanisms for defending and protecting the cells, due to stimulation, by the polysaccharides, of cell communication within the skin explants.

The applicant also carried out an assessment of the effect of the polysaccharides POL.1, POL.2 and POL.3, in aqueous 1% polysaccharide solutions, in reducing the erythema induced by UVB radiation in animals (typically albino guinea pigs). To this end, the animals were exposed to an ultraviolet B light emission source for a time which was defined so as to produce a 2^{nd} order erythematous reaction. The products to be tested were then applied to the areas exposed to the UVB irradiation, with comparison being made with control areas which were exposed to UVB radiation but which did not receive any product. The erythemas were then assessed in accordance with the following gradation scale:
no erythema=0
scarcely visible spots=0.5
mild erythema=1
distinct erythema=2
very visible erythema=3

The results of this assessment are shown in Table VII below:

TABLE VII

| | T + 2 h | T + 5 h | T + 24 h |
|---|---|---|---|
| Control | 11 | 13 | 8 |
| POL. 1 solution | 9 | 8 | 8 |
| POL. 2 solution | 12 | 8 | 6 |
| POL. 3 solution | 13 | 11 | 8 |
| Control | 10 | 11.5 | 4.5 |
| 5% POL. 1 solution | 7 | 4 | 4.5 |
| 5% POL. 2 solution | 10 | 5.5 | 4.5 |
| 5% POL. 3 solution | 10 | 9.5 | 4 |

In conclusion, this study reveals that polysaccharides 1 and 2 have a good soothing effect consequent upon UVB irradiation (“sunburn”). Thus, these 2 molecules substantially reduce the time required for decreasing the erythema which is induced by UVB radiation. On the other hand, polysaccharide 3 is not of any great interest in this study.

In addition, tests of innocuousness were carried out with 0.1% and 1% aqueous solutions of each of the three polysaccharides POL.1, POL.2 and POL.3:
innocuousness test carried out by single oral administration (I.O.A.)—limit assay at the dose of 2000 mg/kg, in accordance with O.J. EEC of 24/04/1984 84/449 L251.
primary cutaneous irritation test (P.C.I.), in accordance with O.J. of 21/02/1982
ocular tolerance test (O.I.), in accordance with O.J. of 10/07/1992

These tests, the results of which are given in Table VIII below, show that, at the concentrations tested, the polysaccharides are perfectly innocuous.

TABLE VIII

| | | P.C.I. - | I.O. - |
|---|---|---|---|
| | I.V.O. | classification | classification |
| POL. 1 - 0.1% | innocuousness | 0.00 - non- | 0.00 - mildly |
| | at the dose of | irritant | irritant |
| | 2000 mg/kg | | |
| POL. 1 - 1% | innocuousness | 0.00 - non- | 0.00 - mildly |
| | at the dose of | irritant | irritant |
| | 2000 mg/kg | | |
| POL. 2 - 0.1% | innocuousness | 0.00 - non- | 0.00 - mildly |
| | at the dose of | irritant | irritant |
| | 2000 mg/kg | | |
| POL. 2 - 1% | innocuousness | 0.00 - non- | 0.00 - mildly |
| | at the dose of | irritant | irritant |
| | 2000 mg/kg | | |
| POL. 3 - 0.1% | innocuousness | 0.00 - non- | 0.00 - mildly |
| | at the dose of | irritant | irritant |
| | 2000 mg/kg | | |
| POL. 3 - 1% | innocuousness | 0.00 - non- | 0.00 - mildly |
| | at the dose of | irritant | irritant |
| | 2000 mg/kg | | |

As the various tests described above demonstrate, the three polysaccharides tested, which are derived from fermenting mesophilic bacteria of hydrothermal origin and which are of differing chemical nature, exhibit significant biological activity. For the skin, this biological activity translates into a variety of protective effects:
protection with regard to microorganisms
protection of the Langerhans cells
soothing action following a “sunburn”

Furthermore, the stimulatory action of the polysaccharides on the production of interleukin 1 by the keratinocytes suggests that these polysaccharides may have wound-healing properties. This is because interleukin 1 participates extremely actively in all the biological processes involved in wound healing:
direct and indirect chemotactic action (production of chemokines): migration of keratinocytes, of monocytes, of lymphocytes, etc.
stimulation of the proliferation of keratinocytes, of fibroblasts, of blood cells, etc.
stimulation of the synthesis of collagens and remodeling of the scar.

All these factors make it possible to recommend the use, in cosmetics, of these polysaccharides for formulating everyday care products for all types of skin, which are directed towards preparing the skin, as well as for formulating specific care products: “anti-aging” products, sunscreen or after-sun products, products for sensitive skins, care and cleansing products for young skins, greasy skins or skins having a tendency to acne, products for damaged hands and feet, aftershave products, hair-treatment products, scalp care products, etc.

The polysaccharide concentration which is recommended for a cosmetic use is between 0.001% and 5%, depending on the sought-after activity and the type of formulation which is used.

The polysaccharides employed can be native (that is to say not physically or chemically modified). They can be depolymerized into fractions of low molecular weight or substantial molecular weight. They can also be modified chemically (sulfated, acidified or nitrogenized). They can be present in lyophilized form, in the form of solutions which are more or less gelatinized, or yet again in encapsulated form.

Naturally, the compositions according to the invention can be present in the form of simple or complex emulsions (water/oil or oil/water creams or milks, triple emulsions, microemulsions and liquid crystal emulsions), of aqueous or oily gels, of aqueous, oily, hydroalcoholic or biphasic lotions, of sticks, or of powders or of any vectorized system (“controlled release” systems or “modulated release” systems). They can be used topically.

Examples of formulations of the cosmetic composition will be described below, by way of non-limiting example:

Moisturizing gel for skins with a tendency to acne

| | |
|---|---|
| Water | QS for 100% |
| 96° alcohol | 5 to 10% |
| Cyclomethicone | 2 to 10% |
| Glycerol | 1 to 5% |
| Sclerotium gum | 0.3 to 0.6% |
| Carbomer | 0.2 to 0.5% |
| Triethanolamine | 0.2 to 0.5% |
| Antimicrobial preservative | 0.1 to 0.7% |
| Perfume | 0.1 to 0.5% |
| PPG-26 buteth-26 & PEG-40 | 0.1 to 0.5% |
| hydrogenated castor oil | |
| Polysaccharide derived from a | 0.001 to 5% |
| fermentation of mesophilic | |
| hydrothermal bacteria | |

Suncream

| | |
|---|---|
| Water | QS for 100% |
| Octyl methoxycinnamate | 5 to 10% |
| Isopropyl lanolate | 2 to 5% |
| Myreth-3 myristate | 2 to 5% |
| PEG-6 stearate & ceteth-20 & | 2 to 5% |
| glyceryl stearate & steareth-20 | |
| Butylmethoxydibenzoylmethane | 1 to 5% |
| Vegetable oil | 1 to 5% |
| Nylon-12 | 1 to 5% |
| Glycerol | 1 to 5% |
| Stearyl dimethicone | 0.5 to 3% |
| Carbomer | 0.2 to 0.6% |
| Triethanolamine | 0.2 to 0.6% |
| Antimicrobial preservative | 0.1 to 0.7% |
| Perfume | 0.1 to 0.5% |
| Tetrasodium EDTA | 0.05 to 0.15% |
| BHT | 0.01 to 0.05% |
| Polysaccharide derived from a | 0.001 to 5% |
| fermentation of mesophilic | |
| hydrothermal bacteria | |

After-sun lotion

| | |
|---|---|
| Water | QS for 100% |
| Dimethicone copolyol | 2 to 5% |
| Propylene glycol | 2 to 5% |
| Myreth-3 myristate | 2 to 5% |
| Vegetable oil | 2 to 5% |
| Mineral oil | 2 to 5% |
| Dimethicone | 2 to 5% |
| Polysorbate 60 | 2 to 3% |
| Sorbitan stearate | 2 to 3% |
| Isopropyl lanolate | 1 to 4% |
| Aluminum starch octenyl-succinate | 1 to 3% |
| Acrylates/C10-30 alkyl acrylate | 0.2 to 0.5% |
| crosspolymer | |
| Triethanolamine | 0.2 to 0.5% |
| Antimicrobial preservative | 0.1 to 0.7% |
| Perfume | 0.1 to 0.5% |
| Tetrasodium EDTA | 0.05 to 0.15% |
| BHT | 0.01 to 0.05% |
| Polysaccharide derived from a | 0.001 to 5% |
| fermentation of mesophilic | |
| hydrothermal bacteria | |

Anti-aging serum

| | |
|---|---|
| Water | QS for 100% |
| Mineral oil | 5 to 10% |
| Dimethicone | 1 to 3% |
| Acrylates/C10-30 alkyl acrylate | 0.2 to 0.5% |
| crosspolymer | |
| Triethanolamine | 0.2 to 0.5% |
| Antimicrobial preservative | 0.1 to 0.7% |
| Perfume | 0.1 to 0.5% |
| Polysaccharide derived from a | 0.001 to 5% |
| fermentation of mesophilic | |
| hydrothermal bacteria | |

Regulating shampoo

| | |
|---|---|
| Water | QS for 100% |
| Sodium laureth sulfate | 10 to 20% |
| Cocamidopropyl betaine | 5 to 15% |
| Caprylyl/capryl glucoside | 5 to 10% |
| Cocamide DEA | 2 to 5% |
| Acrylate/steareth-20 methacrylate | 1 to 4% |
| copolymer | |
| Glycerol | 1 to 3% |
| PEG-120 methyl glucose dioleate | 0.5 to 2% |
| Perfume | 0.2 to 1% |
| Antimicrobial preservative | 0.1 to 0.7% |
| Tetrasodium EDTA | 0.05 to 0.15% |
| Polysaccharide derived from a | 0.001 to 5% |
| fermentation of mesophilic | |
| hydrothermal bacteria | |

1. A process for improving the immune system for defending and protecting the cells of a human skin, said process comprising a topically applying a cosmetic composition comprising exopolysaccharides generated by mesophilic bacteria obtained from deep-sea hydrothermal vents located beyond a depth of 2500 meters, said exopolysaccharide consisting of polymers having different neutral sugar/acid ratios with a basic monomeric unit representing 4 to 10 residues and with molecular weight comprised between 100 000 and 1 000 000 Daltons, said process having the effect of stimulating keratinocytes by said exopolysaccharides to induce the expression of interleukin 1-α so as to arouse the immune defense system of the skin which is then able to react more vigorously and rapidly to external.

2. The process as claimed in claim 1, wherein the exopolysaccharide in said cosmetic composition is between 0.001% and 5%.

3. The process as claimed in claim 1, wherein the exopolysaccharides are native polysaccharides.

4. The process as claimed in claim 1, wherein the exopolysaccharides are depolymerized into fractions of different molecular weight.

5. A cosmetic process as claimed in claim 1, wherein the exopolysaccharide is subjected to a chemical reaction so that they are modified by at least one of sulphated, acidified and nitrogenated.

6. The process as claimed in claim 1, wherein the exopolysaccharide is presented in lyophilized form, in solution form, or in encapsulated form.

7. The process as claimed in claim 1, wherein said cosmetic composition is presented in the form of simple or complex emulsions, of aqueous or oily gels, of aqueous, oily, hydroalcoholic or biphasic lotions, of sticks, of powders or of a vectorized system.

8. The process as claimed in claim 1, wherein said cosmetic composition is applied as everyday cosmetic skin care products.

9. The process as claimed in claim 1, wherein said cosmetic composition is applied as an anti-aging care products.

10. The process as claimed in claim 1, wherein said cosmetic composition is applied as sunscreen or after-sun products.

11. The process as claimed in claim 1, wherein said cosmetic composition is applied as products for sensitive skins.

12. The process as claimed in claim 1, wherein said cosmetic composition is applied as care and cleansing products for young skins, greasy skins or skins having a tendency to acne.

13. The process as claimed in claim 1, wherein said cosmetic composition is applied as cosmetic products for hands and feet.

14. The process as claimed in claim 1, wherein said cosmetic composition is applied as aftershave products.

15. The process as claimed in claim 1, wherein said cosmetic composition is applied as hair-treatment products.

16. The process as claimed in claim 1, wherein said cosmetic composition is applied as scalp care products.

17. The process as claimed in claim 1, wherein said cosmetic composition consists of moisturizing gel for skins having a tendency to acne, which at least possesses the following formula:

| | |
|---|---|
| Water | QS for 100% |
| Ethanol 96% in water | 5 to 10% |
| Cyclomethicone | 2 to 10% |
| Glycerol | 1 to 5% |
| Sclerotium gum | 0.3 to 0.6% |
| Carbomer | 0.2 to 0.5% |
| Triethanolamine | 0.2 to 0.5% |
| Antimicrobial preservative | 0.1 to 0.7% |
| Perfume | 0.1 to 0.5% |
| PPG-26 buteth-26 & PEG-40 hydrogenated | 0.1 to 0.5% |
| castor oil | |
| Exopolysaccharide consisting of | 0.001 to 5% |
| polymers having a molecular weight | |
| comprised between 500 000 and 1 000 000 | |
| Daltons and having chains which consist | |
| exclusively of neutral and/or acid sugars, | |
| with basic monomeric unit representing 4 | |
| to 10 residues, said exopolysaccharide | |
| being produced by Alteromonas providing | |
| from deep-sea hydrothermal vents located | |
| beyond a depth of 2500 meters. | |

18. The process as claimed in claim 1, wherein the cosmetic composition consist of a sun cream, having the following formula:

| | |
|---|---|
| Water | QS for 100% |
| Octyl methoxycinnamate | 5 to 10% |
| Isopropyl lanolate | 2 to 5% |
| Myreth-3 myristate | 2 to 5% |
| PEG-6 stearate & ceteth-20 & glyceryl | 2 to 5% |
| stearate & steareth-20 | |
| Butylmethoxydibenzoylmethane | 1 to 5% |
| Vegetable oil | 1 to 5% |
| Nylon-12 | 1 to 5% |
| Glycerol | 1 to 5% |
| Stearyl dimethicone | 0.5 to 3% |
| Carbomer | 0.2 to 0.6% |
| Triethanolamine | 0.2 to 0.6% |
| Antimicrobial preservative | 0.1 to 0.7% |
| Perfume | 0.1 to 0.5% |
| Tetrasodium EDTA | 0.05 to 0.15% |
| BHT 0.01 to 0.05% | |
| Exopolysaccharide consisting of | 0.001 to 5% |
| polymers having a molecular weight | |
| comprised between 500 000 and 1 000 000 | |
| Daltons and having chains which consist | |
| exclusively of neutral and/or acid sugars, | |
| with basic monomeric unit representing 4 | |
| to 10 residues, said exopolysaccharide | |
| being produced by Alteromonas providing | |
| from deep-sea hydrothermal vents located | |
| beyond a depth of 2500 meters. | |

19. The process as claimed in claim 1, wherein the cosmetic composition consists of an after-sun lotion, having the following formula:

| | |
|---|---|
| Water | QS for 100% |
| Dimethicone copolyol | 2 to 5% |
| Propylene glycol | 2 to 5% |
| Myreth-3 myristate | 2 to 5% |
| Vegetable oil | 2 to 5% |
| Mineral oil | 2 to 5% |
| Dimethicone | 2 to 5% |
| Polysorbate 60 | 2 to 3% |
| Sorbitan stearate | 2 to 3% |
| Isopropyl lanolate | 1 to 4% |
| Aluminum starch octenyl-succinate | 1 to 3% |
| Acrylates/C10-30 alkyl acrylate | 0.2 to 0.5% |
| crosspolymer | |
| Triethanolamine | 0.2 to 0.5% |
| Antimicrobial preservative | 0.1 to 0.7% |
| Perfume | 0.1 to 0.5% |
| Tetrasodium EDTA | 0.05 to 0.15% |
| BHT | 0.01 to 0.05% |
| Exopolysaccharide consisting of | 0.001 to 5% |
| polymers having a molecular weight | |
| comprised between 500 000 and 1 000 000 | |
| Daltons and having chains which consist | |
| exclusively of neutral and/or acid sugars, | |
| with basic monomeric unit representing 4 | |
| to 10 residues, said exopolysaccharide | |
| being produced by Alteromonas providing | |
| from deep-sea hydrothermal vents located | |
| beyond a depth of 2500 meters. | |

20. The process as claimed in claim 1, wherein the cosmetic composition consists of an anti-aging serum, having the following formula:

| | |
|---|---|
| Water | QS for 100% |
| Mineral oil | 5 to 10% |
| Dimethicone | 1 to 3% |
| Acrylates/C10-30 alkyl acrylate | 0.2 to 0.5% |
| crosspolymer | |
| Triethanolamine | 0.2 to 0.5% |
| Antimicrobial preservative | 0.1 to 0.7% |
| Perfume | 0.1 to 0.5% |
| Exopolysaccharide consisting of | 0.001 to 5% |
| polymers having a molecular weight | |
| comprised between 500 000 and 1 000 000 | |
| Daltons and having chains which consist | |
| exclusively of neutral and/or acid sugars, | |
| with basic monomeric unit representing 4 | |
| to 10 residues, said exopolysaccharide | |
| being produced by Alteromonas providing | |
| from deep-sea hydrothermal vents located | |
| beyond a depth of 2500 meters. | |

21. The process as claimed in claim 1, wherein the cosmetic composition consists of a regulating shampoo, having the following formula:

| | |
|---|---|
| Water | QS for 100% |
| Sodium laureth sulfate | 10 to 20% |
| Cocamidopropyl betaine | 5 to 15% |
| Caprylyl/capryl glucoside | 5 to 10% |
| Cocamide DEA | 2 to 5% |
| Acrylate/steareth-20 methacrylate | 1 to 4% |
| copolymer | |
| Glycerol | 1 to 3% |
| PEG-120 methyl glucose dioleate | 0.5 to 2% |
| Perfume | 0.2 to 1% |
| Antimicrobial preservative | 0.1 to 0.7% |
| Tetrasodium EDTA | 0.05 to 0.15% |
| Exopolysaccharide consisting of | 0.001 to 5% |
| polymers having a molecular weight | |
| comprised between 500 000 and 1 000 000 | |
| Daltons and having chains which consist | |
| exclusively of neutral and/or acid sugars, | |
| with basic monomeric unit representing 4 | |
| to 10 residues, said exopolysaccharide | |
| being produced by Alteromonas providing | |
| from deep-sea hydrothermal vents located | |
| beyond a depth of 2500 meters. | |
